(19) United States
(12) Patent Application Publication
(10) Pub. No.: US 2021/0401308 A1
(43) Pub. Date: Dec. 30, 2021

(54) IMPEDANCE PLETHYSMOGRAM USING OPTICAL GATING SIGNAL AND STRUCTURE WITH INTEGRATED ELECTRODES AND OPTICAL SENSOR

(71) Applicant: MGI, LLC., Ridgefield, NJ (US)

(72) Inventor: Lloyd A. Marks, Westfield, NJ (US)

(21) Appl. No.: 17/364,826

(22) Filed: Jun. 30, 2021

Related U.S. Application Data
(60) Provisional application No. 63/046,587, filed on Jun. 30, 2020.
(60) Provisional application No. 63/170,690, filed on Apr. 5, 2021.

(51) Int. Cl. A61B 5/0295; A61B 5/024; A61B 5/1455; A61B 5/00

(52) U.S. Cl. CPC A61B 5/0295; A61B 5/02427; A61B 5/6824; A61B 5/02438; A61B 5/1455

(57) ABSTRACT

A structure for a plethysmogram and a method for obtaining an optical gating signal using a plethysmogram are disclosed. The structure includes: a backing; a plurality of electrodes mounted on the backing; a reflectance (or transmission) optical sensor mounted on the backing; and a cable that connects the plurality of electrodes and the reflectance optical sensor to a pulse flowmeter. In an embodiment, “look-back” software is employed to obtain an optical gating signal for averaging the impedance waveforms associated with each heartbeat.

### CROSS REFERENCE TO RELATED APPLICATION(S)

This application claims priority to U.S. Provisional Patent Application Ser. No. 63/046,587, filed on Jun. 30, 2020 and U.S. Provisional Patent Application Ser. No. 63/170,690, filed on Apr. 5, 2021, both of which are hereby incorporated herein by reference in their entireties.

### GOVERNMENT SPONSORSHIP

None

### FIELD OF THE INVENTION

Embodiments are in the field of systems and methods for plethysmography. More particularly, embodiments disclosed herein relate to systems and methods for plethysmography, including an impedance plethysmogram using optical gating signal and structure with integrated electrodes and optical sensor, which enables simpler, expedient, and accurate procedures.

### BACKGROUND OF THE INVENTION

Pulse volume is the small change in the volume of a limb segment that occurs with each heartbeat and can be measured with the devices described in U.S. Pat. No. 4,548,211 “Computer Assisted Admittance Plethysmograph” and U.S. Pat. No. 10,231,635 “Impedance Plethysmograph Using Concurrent Processing.” These devices use “selective signal averaging” in which the first step is to detect a heartbeat from the QRS complex of the electrocardiogram (ECG). A sampling window is then opened during which time the impedance (or equivalently the admittance) waveform associated with that cardiac cycle is captured. That single waveform is accepted or rejected for averaging based upon specific noise criteria and, if acceptable, is included in a running average with prior and successive beats. About 30 cardiac cycles provides a reasonably reproducible composite impedance waveform, from which a pulse volume waveform can be derived. The amplitude of the pulse volume waveform is measured with a computer algorithm and this measurement is designated as the “pulse volume.” The pulse volume multiplied by the heart rate is the “pulse flow” and the machine that these measurements are made with is called a “Pulse Flowmeter.” The term “photoplethysmogram” and phrase “pulse oximeter” are often used interchangeably. A photoplethysmogram merely produces and measures light of one or more frequencies. Red is used in this invention because red light bounces off of red blood cells. A pulse oximeter uses 2 beams of red light, one of which bounces off oxygenated red blood cells and the other of which bounces off of deoxygenated red blood cells. The ratio of the intensity of the red beams determines the oxygen saturation (the percent of hemoglobin which is oxygenated) with a look-up table. Pulse oximeters and optical plethysmographs can be either transmission or reflectance types. The choice of which to use is at least, in part, economic, as pulse oximeters are mass produced and inexpensive. Either one will work for the application described below.

First, in order to perform signal averaging it is necessary to have a “gating” signal, that is, a specific known point in time that occurs in a fixed time relationship with the waveform that is to be averaged. For cardiac activity, the QRS complex provides an ideal gating signal. However, the device must either be electronically integrated with a patient's ECG monitor or additional electrodes must be placed on the patient's torso and connected to the Pulse Flowmeter. Furthermore, an ECG waveform can be noisy, resulting in false positive or false negative QRS detections. When that occurs, the captured waveform does not occur in a fixed time relationship with the other captured waveforms and the average is corrupted.

Second, the current instrument uses 4 stretchable circumferential electrodes 100 as described in U.S. Pat. No. 8,019,401 “Stretchable Electrode and Method of Making Physiological Measurements” which are mounted on a stretchable neoprene base and held on to the arm with a hook-and-loop fastener (see FIG. 1). The two outer electrodes are used to pass a 40 KHz, 1 milliamp current through the limb segment and the two inner electrodes are used to measure the voltage resulting from that current. The limb is modelled as a cylinder and the assumption is made that the current has reasonably dispersed across the limb by the time it passes the voltage sensing electrodes. That allows calculation of the volume change from the impedance change (as described in the aforementioned patents). One problem is that a circumferential electrode is difficult to manufacture and cumbersome to apply to the limb/arm. Also, they require a separate set of cables, other than the ECG leads, connected to the Pulse Flowmeter machine. Another problem is that a disposable version of the electrodes uses a conductive adhesive (such as a hydrogel) to hold it on to the arm and the electrodes (with the adhesive applied) may get irreversibly tangled during application on the arm if not done very carefully.

Thus, it is desirable to provide a plethysmogram that is able to overcome the above disadvantages, and which enables simpler, expedient, and accurate procedures.

Advantages of the present invention will become more fully apparent from the detailed description of the invention hereinbelow.

### SUMMARY OF THE INVENTION

Embodiments are directed to a structure for a plethysmogram, the structure including: a backing; a plurality of electrodes mounted on the backing; a reflectance optical sensor mounted on the backing; and a cable that connects the plurality of electrodes and the reflectance optical sensor to a pulse flowmeter.

Embodiments are also directed to a method for obtaining an optical gating signal using a plethysmogram. The method includes: obtaining a heartbeat waveform, wherein the heartbeat has a heart rate associated therewith; determining the onset time based on the heart rate; determining the offset time based on the onset time; and selecting an impedance waveform from the onset time to the offset time, wherein the impedance waveform functions as an optical gating signal.

Additional embodiments and additional features of embodiments for the structure for a plethysmogram and method for obtaining an optical gating signal using a plethysmogram are described below and are hereby incorporated into this section.

### BRIEF DESCRIPTION OF THE DRAWINGS

The foregoing summary, as well as the following detailed description, will be better understood when read in conjunction with the appended drawings. For the purpose of illustration only, there is shown in the drawings certain embodiments. It is understood, however, that the inventive concepts disclosed herein are not limited to the precise arrangements and instrumentalities shown in the figures. The detailed description will refer to the following drawings in which like numerals, where present, refer to like items.

FIG. 1A is a drawing illustrating a structure including four stretchable circumferential electrodes mounted on a stretchable neoprene base;

FIG. 1B is a drawing illustrating the structure, shown in FIG. 1A, mounted on a limb (such as an arm) of a patient;

FIG. 2 is a drawing illustrating a structure including four spot electrodes and a reflectance pulse detector (i.e., a type of reflectance optical sensor);

FIG. 3 is a drawing illustrating a structure including four semi-circumferential rectangular electrodes/contacts and a reflectance pulse oximeter/detector;

FIG. 4 is a drawing illustrating a structure including four semi-circumferential rectangular electrodes/contacts and a reflectance pulse oximeter, in a tapered configuration;

FIG. 5 is a drawing illustrating a structure including four semi-circumferential rectangular electrodes/contacts and a reflectance pulse oximeter, in a paired configuration;

FIG. 6 is a drawing illustrating a structure including four semi-circumferential rectangular electrodes/contacts, a transmission optical light source, and a receiving optical light sensor;

FIG. 7 is a drawing illustrating a structure including four semi-circumferential rectangular electrodes/contacts intended for use with a finger/toe/earlobe/etc. transmission pulse oximeter (or optical plethysmograph) to provide the gating signal;

FIG. 8 is a plot illustrating waveforms used in look-back software for the embodiments of the invention which use an optical gating signal; and

FIG. 9 is a flowchart illustrating an embodiment of a method for obtaining an optical gating signal using a plethysmogram.

### DETAILED DESCRIPTION OF THE INVENTION

It is to be understood that the figures and descriptions of the present invention may have been simplified to illustrate elements that are relevant for a clear understanding of the present invention, while eliminating, for purposes of clarity, other elements found in a typical plethysmograph or typical method of using a plethysmograph. Those of ordinary skill in the art will recognize that other elements may be desirable and/or required in order to implement the present invention. However, because such elements are well known in the art, and because they do not facilitate a better understanding of the present invention, a discussion of such elements is not provided herein. It is also to be understood that the drawings included herewith only provide diagrammatic representations of the presently preferred structures of the present invention and that structures falling within the scope of the present invention may include structures different than those shown in the drawings. Reference will now be made to the drawings wherein like structures are provided with like reference designations.

Before explaining at least one embodiment in detail, it should be understood that the inventive concepts set forth herein are not limited in their application to the construction details or component arrangements set forth in the following description or illustrated in the drawings. It should also be understood that the phraseology and terminology employed herein are merely for descriptive purposes and should not be considered limiting.

It should further be understood that any one of the described features may be used separately or in combination with other features. Other invented devices, systems, methods, features, and advantages will be or become apparent to one with skill in the art upon examining the drawings and the detailed description herein. It is intended that all such additional devices, systems, methods, features, and advantages be protected by the accompanying claims.

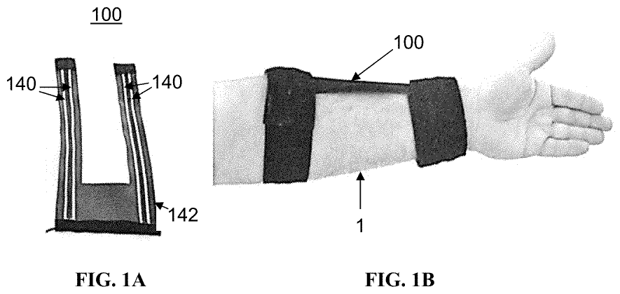

FIG. 1A is a drawing illustrating a structure 100 including four stretchable circumferential electrodes 140 mounted on a stretchable neoprene base 42.

FIG. 1B is a drawing illustrating the structure 100 shown in FIG. 1A mounted on a limb (such as an arm 1) of a patient.

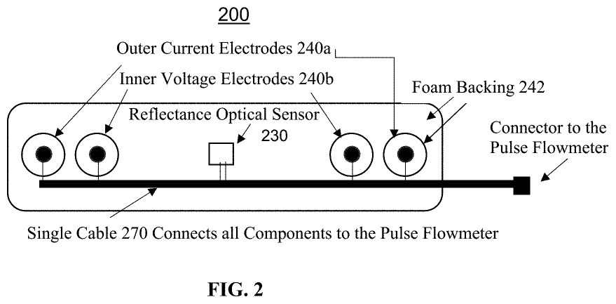

FIG. 2 is a drawing illustrating a structure 200 including four spot electrodes (outer current electrodes 240a and inner voltage electrodes 240b) and a reflectance pulse detector (i.e., a reflectance optical sensor 230). The structure 200 includes a foam backing 242 for the electrodes to mount thereon. The electrodes 240a, 240b and optical sensor 230 are connected to a cable which connects to a pulse flowmeter (not shown).

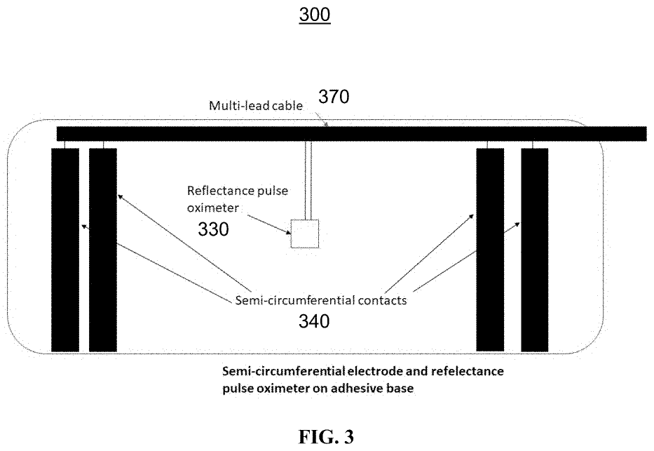

FIG. 3 is a drawing illustrating a structure 300 including four semi-circumferential rectangular electrodes/contacts 340 and a reflectance pulse oximeter/detector/sensor 330. The electrodes 340 and reflectance pulse oximeter 330 are connected to a single cable 370 which connects to a pulse flowmeter (not shown).

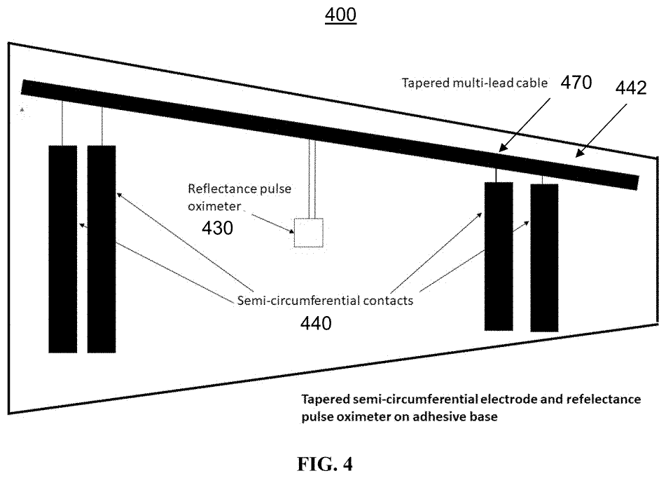

FIG. 4 is a drawing illustrating a structure 400 including four semi-circumferential rectangular electrodes/contacts 440 and a reflectance pulse oximeter 430, in a tapered configuration. The foam backing 442 is also in a tapered configuration. The electrodes 440 and reflectance pulse oximeter 430 are connected to a single cable 470 (which is angled with respect to the electrodes 440, to correspond to the tapering of the foam backing 442) which connects to a pulse flowmeter (not shown).

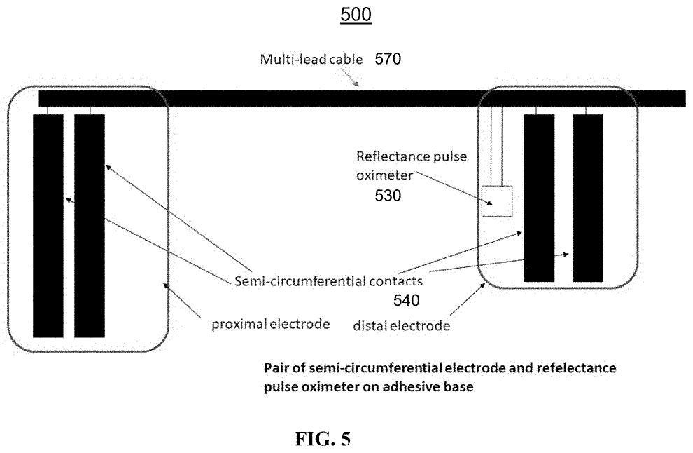

FIG. 5 is a drawing illustrating a structure 500 including four semi-circumferential rectangular electrodes/contacts 540 and a reflectance pulse oximeter 530, in a paired configuration. That is, the two electrodes 540 on the left side of the figure are part of the proximal electrode, and the two electrodes 540 on the right side of the figure are part of the distal electrode. The electrodes 540 and reflectance pulse oximeter 530 are connected to a single cable 570 which connects to a pulse flowmeter (not shown).

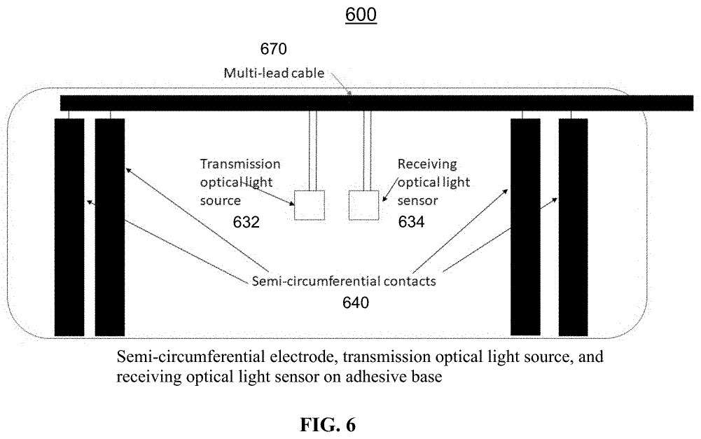

FIG. 6 is a drawing illustrating a structure 600 including four semi-circumferential rectangular electrodes/contacts 640, a transmission optical light source 632, and a receiving optical light sensor 634. The electrodes 640, transmission optical light source 632, and receiving optical light sensor 634 are all connected to a single cable 670 which connects to a pulse flowmeter (not shown).

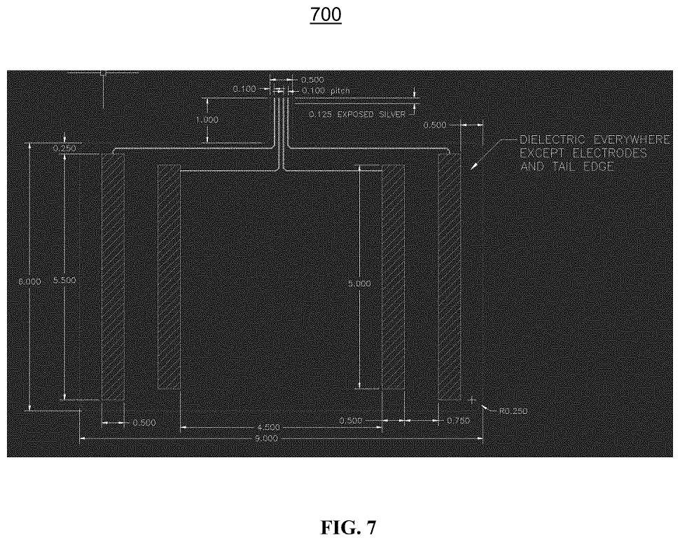

FIG. 7 is a drawing illustrating a structure 700 including four semi-circumferential rectangular electrodes/contacts intended for use with a finger/toe/earlobe/etc. transmission pulse oximeter (or optical plethysmograph) which provides the gating signal. There is a dielectric in all areas except for the electrodes and tail edge.

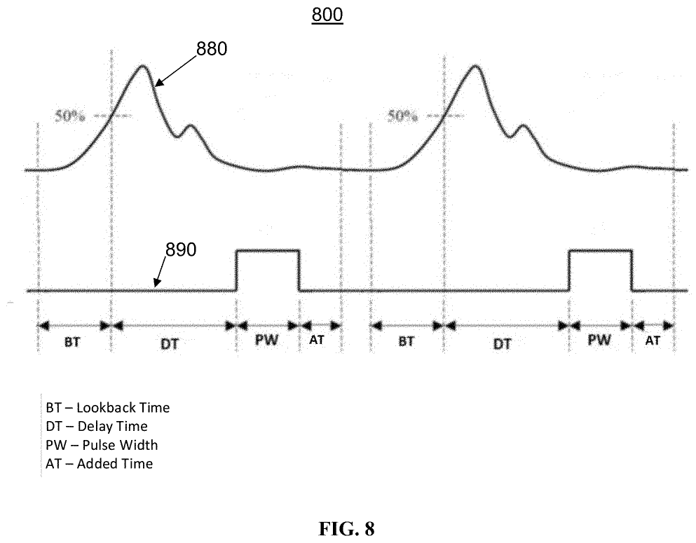

FIG. 8 is a plot 800 illustrating waveforms used in look-back software to be used in conjunction with any of the structures shown in FIGS. 2-7. In FIG. 8, the top waveform 880 represents the impedance plethysmogram associated with a heartbeat detected by the pulse oximeters (FIGS. 2-6) or the receiving optical light sensor 634 (FIG. 6). The bottom waveform 890 in FIG. 8 represents the gating signal.

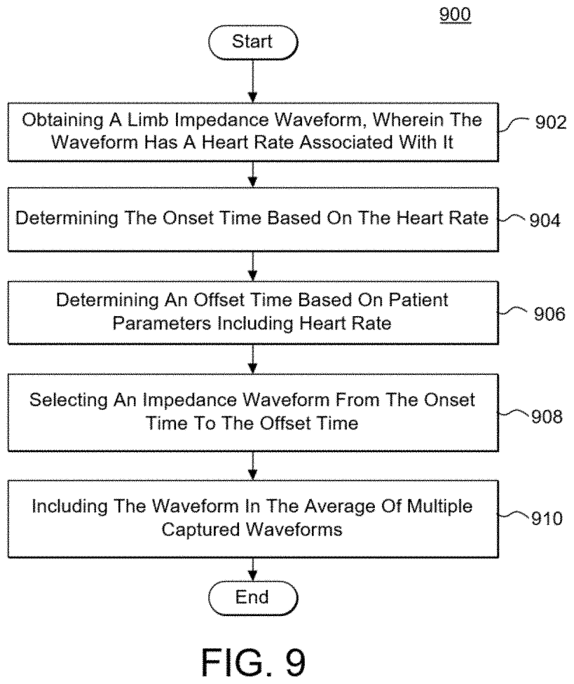

FIG. 9 is a flowchart illustrating an embodiment of a method 900 for obtaining an optical gating signal using a plethysmogram.

In this invention, the aforementioned problems regarding the use of the electrocardiogram to obtain a gating signal are solved by replacing the ECG with an optical sensor that provides a photoplethysmography waveform (which occurs in a fixed time relationship with both the QRS complex and the limb segment impedance waveform) to provide the gating signal. The photoplethysmogram may be obtained from a transmittance device such as the pulse oximeters that are routinely used for patient monitoring or with a reflectance device such as that manufactured by Rohm, which is commonly used in devices like the Fitbit® or Apple Watch to monitor heart rate. The Rohm device has a separate circuit board for signal conditioning and pulse detection. That circuit board can be positioned inside the Pulse Flowmeter box. A finger (or toe) transmittance photoplethysmograph can impede a patient's ability to use their hands or feet and requires a separate cable to the box which may be a source of tangling or obstruction. Other advantages of using a photoplethysmography or reflectance pulse oximeter device are that it can be integrated with the electrodes into a single structure so that it does not impede the patient's ability to use their hands or feet and that all information, both the optical signal and the impedance plethysmography, can be transmitted to the Pulse Flowmeter with a single cable. In a medical environment where a patient may be attached to multiple tubes and wires, this is a very desirable characteristic. An alternate method of optical pulse detection is laser doppler flowmetry which measures the spectral broadening that takes place when monochromatic laser light interacts with moving red blood cells. Currently, these devices are primarily used to check for tissue viability when deciding where to position a skin graft, typically in a patient who has had third degree burns.

One important difference between using the ECG and an optical pulse detector is that in the former, the gating signal occurs before the impedance waveform whereas in the latter, the gating signal occurs during the impedance waveform. Therefore, to capture the entire impedance waveform associated with a heartbeat that has been detected optically, the impedance data is continuously saved and “looked back” upon. In other words, if the QRS occurs at time=0 and the optical pulse isn't detected until 150 milliseconds later, the impedance waveform data from time=0 is retrieved and included in the full impedance waveform associated with that specific heartbeat.

To solve the aforementioned other problems, i.e., those related to using quadripolar fully circumferential electrodes, 4 spot or partially elongated electrodes can be used. The limb segment can no longer be modelled as a cylinder so the impedance change (and therefore the volume change) that occurs in the current path between the outer spot electrodes is measured and that will not be a cylinder. Experimentation has revealed that pulse volume and pulse flow measurements made in the calf are similar whether circumferential or non-circumferential electrodes are used.

With reference to FIG. 2, in one embodiment of this invention described herein, 4 spot electrodes and a reflectance pulse detector are integrated into a single structure (FIG. 2) which can be placed on and attached to a patient's limb segment. Information from all five of these structures can be passed to the Pulse Flowmeter through a single cable. Furthermore, as the distance between the inner voltage electrodes is used in the formula that computes volume changes from impedance changes (and the inner electrodes may be spaced differently in different sized composite structures), that distance information can also be conveyed to the Pulse Flowmeter box as described in U.S. Pat. No. 7,945,318 “Peripheral Impedance Plethysmography Electrode and System with Detection of Electrode Spacing” through the single cable connecting the electrode to the Pulse Flowmeter.

An embodiment of the single structure containing the 4 spot electrodes and the reflectance pulse detector is shown in FIG. 2. It may be desirable to have all the components mounted on a foam base. It may also be desirable to have an adhesive with a peel-off coating covering the patient side of the structure so that a simple, single, peel off operation can be followed by application of the structure to the patient. Such a structure could be disposable, a desirable characteristic in many hospitals and ambulatory surgical centers.

In another embodiment, if non-circumferential electrodes are desired, but spot electrodes are considered too small, 2 pairs of rectangular contacts as shown in FIG. 3 can be used. The contacts are sized so that they provide a larger surface area of contact with the limb, though not fully circumferential.

As the closer the electrodes are to being circumferential, the more accurate the measurement is and because fully circumferential electrodes are difficult to manipulate and manufacture, an intermediary solution is to use semi-circumferential electrodes as described below.

Instead of using spot electrodes as shown in FIG. 2, a wider electrode with semi-circumferential rectangular contacts may contact the skin as shown in FIG. 3.

Because the forearm and calf taper, it may be desirable to configure a tapering electrode as shown in FIG. 4. The larger diameter proximal end would go around the proximal larger part of the forearm or calf and the distal smaller diameter end would go around the smaller diameter part of the forearm or calf.

Alternatively, and perhaps, preferably, the contacts could be divided into two electrodes, a proximal larger diameter one and a distal smaller diameter one as shown in FIG. 5. That would have the advantages that it would not “buckle” on a tapering limb, would be easier to manufacture and could be stored as a pair in a smaller package. The distance between the inner electrodes must be known as it is an input into the computer algorithm that determines pulse volume from changes in limb impedance, so that could be fixed by the distance of the portion of multi-lead cable between the inner electrodes. Alternatively, as described in our U.S. Pat. No. 7,945,318, the distance between the inner electrodes can be communicated to the Pulse Flowmeter if different distances are desired or required (such as making an electrode or electrodes for a baby or child). One disadvantage would be that 2 electrodes would have to be placed on the limb instead of one.

It will be apparent to one skilled in the art that a transmission optical plethysmograph can be used instead of a reflectance optical plethysmograph as long as the light source is strong enough to be received by the optical sensor after it interacts with blood cells. Such an arrangement is shown in FIG. 6.

It will also be apparent to one skilled in the art that the semicircular electrode need not incorporate an optical sensor. The plethysmography signal may be derived from a finger, ear lobe or toe transmission pulse oximeter. In our upcoming clinical trials we plan to use an electrode without optical sources or sensors and a finger/toe/earlobe/etc. transmission pulse oximeter (or optical plethysmograph) to provide the gating signal. That feature will likely be incorporated to eliminate one of the electrical cables that go to the patient. The plan for a non-optical electrode is shown in FIG. 7.

The electrode in FIG. 7 is to be placed on the back of the calf (probably) but can also be placed on the arm without integrated optical sensors. The big difference between using the QRS complex and the optical plethysmograph to provide the gating signal is that the former occurs before the pulse wave and the latter occurs during the pulse. Therefore, to visualize and measure the full averaged pulse wave, including the part of the waveform that preceded the pulse oximeter waveform, we use “look-back” software which is described hereinafter:

The optical pulse is detected during the pulse wave as opposed to the QRS complex of the ECG which occurs before the pulse wave. To average and otherwise process the complete plethysmographic pulse waveforms, the algorithm must look back before the rise of each pulse from its baseline until the optical pulse is detected. That may be called the Before Time (BT). The software must also include the trailing time after the pulse settles. That may be referred to as After Time (AT). The moving average of the waveforms includes the BT through and including the AT. DT (or delay time, is the time from when the pulse has achieved 50% of its maximum amplitude until the gating pulse (PW) is generated. It will be apparent to one skilled in the art that these time intervals may be adapted to suit different heart rates and pulse amplitudes. A typical time for BT+DT+PW+AT is one second (heart rate of 60). Also, it will be apparent to one skilled in the art that other methods of pulse detection (such as exceeding a dV/dt threshold may be employed. BT and AT are typically 100 msec but may be modified, typically in response to heart rate changes.

As shown in the FIG. 8, the algorithm is buffering the impedance plethysmographic signals in real-time, then when calculating the moving average, the software “looks back” and synchronizes the pulse cycles for averaging based on the optical pulse generated by a photoplethysmogram or pulse oximeter.

All the data from the plethysmogram is stored in memory. The look-back algorithm (embodied in software) is looking back at the data stored in the buffer that contains the impedance plethysmographic data. In one example, the algorithm looks back preferably 100 ms before the detection of the pulse, and 100 ms after the pulse is terminated. In other words, the algorithm picks a lookback time frame based on heart rate. If the heart rate is slow (i.e., a typical/normal heart rate), like 60 ms, the algorithm may look back 100 ms, for example. In another example, if the heart rate was 140, the look-back may be reduced to 90 ms. The lookback time is arbitrary, but the timing is such that it uses as much of the horizontal time space as possible (without capturing the next pulse volume plethysmographic waveform). Thus, if someone's heart rate was 200 per minute (i.e., very fast), if the forward and backward windows are too long, then the algorithm ends up looking at the beat that occurred before and the beat that occurred after the waveform of interest. Optionally, the configuration might independently have one lookup time before and a different lookup time after, and that could be adapted based on heart rate or other parameters. During operation of the system, the optical sensor (pulse oximeter) picks up the pulse using, for example, any of the structures in FIGS. 2-7 described in this application. And the algorithm is looking from some fixed time interval before the pulse is detected by the pulse oximeter to some fixed time interval after the pulse is detected by the pulse oximeter.

The above process/algorithm is incorporated via software and is executed on a processor of a computer system such as a laptop, table, PC, or mobile device. The start and end points are picked and the waveforms are averaged. An important factor is that the look-back time must remain the same for each interval before the heartbeat is detected by pulse oximetry. The pulse oximetry detection occurs during the middle portion of the pulse, whereas, in significant contrast, the QRS complex of the ECG occurs before the pulse. Advantages of the detection of the pulse during the middle portion of the pulse are that pulse oximetry may provide a more stable gating signal and that the pulse oximeter (or optical plethysmogram) can be physically integrated into the electrode, whereas ECG electrodes cannot. One important advantage with the present designs is that when a pulse oximeter or optical plethysmograph are integrated into the electrode (such as in the embodiments of FIGS. 2-6), there is one less lead going to the patient. As mentioned above, these configurations do not use an ECG. So, the only necessary cable going to the patient is to the electrode.

Main steps of the “look-back” process include:
obtaining a limb impedance waveform, wherein the waveform has a heart rate associated with it;
determining the onset time based on the heart rate;
determining an offset time based on patient parameters including heart rate;
selecting an impedance waveform from the onset time to the offset time; and
including the waveform in the average of multiple captured waveforms, i.e., if it meets suitable noise criteria.

The above process evokes a response. More specifically, the heartbeat is evoking the response. In this case, we are detecting the heartbeat with a pulse oximeter. Or it could be detected with an ECG. Either way, the response is the impendence waveform. By averaging using the above technique, most or all of random noise is eliminated.

Embodiments are directed to a structure for a plethysmogram, the structure including: a backing; a plurality of electrodes mounted on the backing; a reflectance (or transmission) optical sensor mounted on the backing; and a cable that connects the plurality of electrodes and the reflectance optical sensor to a pulse flowmeter.

In an embodiment, the reflectance optical sensor is a reflectance pulse oximeter.

In another embodiment, the optical sensor is an optical plethysmograph including a light source and an optical sensor.

In an embodiment of the method, the plurality of electrodes are spot electrodes.

In an embodiment of the method, the plurality of electrodes are semi-circumferential electrodes.

In an embodiment of the method, the backing is tapered.

In an embodiment of the method, the plurality of electrodes comprise two electrodes as part of a proximal electrode, and another two electrodes are part of a distal electrode.

In an embodiment of the method, the method further includes a transmission optical light source, and the reflectance optical sensor includes a receiving optical light sensor.

In an embodiment of the method, the method further includes deriving the gating signal from an impedance plethysmogram.

Embodiments are also directed to a method 900 for obtaining an optical gating signal using a plethysmogram. With reference to FIG. 9, the method 900 includes: obtaining a limb impedance waveform, wherein the waveform has a heart rate associated with it (block 902); determining the onset time based on the heart rate (or other factors) (block 904); determining an offset time based on patient parameters including heart rate (block 906); selecting an impedance waveform from the onset time to the offset time (block 908); and including the waveform in the average of multiple captured waveforms, i.e., if it meets suitable noise criteria (block 910).

In an embodiment of the method 900, the heartbeat optical plethysmogram or pulse oximetry waveform is used to provide a gating signal.

In an embodiment of the method 900, the step of determining the onset time looks at a previous rise of the heartbeat waveform from its baseline.

In an embodiment of the method 900, the step of determining the offset time looks at a subsequent trailing time after the heartbeat waveform settles back down from the previous rise.

In an embodiment of the method 900, the step of obtaining a gating signal is performed via an optical plethysmogram or pulse oximeter (which may be a transmission or reflectance pulse oximeter). In one embodiment, the plethysmogram may include a reflectance pulse oximeter to detect the heartbeat waveform in the step of obtaining. In another embodiment, the optical gating signal and a signal representing the heartbeat waveform may be transmitted via a single cable.

The method steps in any of the embodiments described herein are not restricted to being performed in any particular order. Also, structures or systems mentioned in any of the method embodiments may utilize structures or systems mentioned in any of the device/system embodiments. Such structures or systems may be described in detail with respect to the device/system embodiments only but are applicable to any of the method embodiments.

Features in any of the embodiments described in this disclosure may be employed in combination with features in other embodiments described herein, such combinations are considered to be within the spirit and scope of the present invention.

The contemplated modifications and variations specifically mentioned in this disclosure are considered to be within the spirit and scope of the present invention.

More generally, even though the present disclosure and exemplary embodiments are described above with reference to the examples according to the accompanying drawings, it is to be understood that they are not restricted thereto. Rather, it is apparent to those skilled in the art that the disclosed embodiments can be modified in many ways without departing from the scope of the disclosure herein. Moreover, the terms and descriptions used herein are set forth by way of illustration only and are not meant as limitations. Those skilled in the art will recognize that many variations are possible within the spirit and scope of the disclosure as defined in the following claims, and their equivalents, in which all terms are to be understood in their broadest possible sense unless otherwise indicated.

1. A structure for a plethysmogram, the structure comprising:
a backing;
a plurality of electrodes mounted on the backing;
a reflectance optical sensor mounted on the backing; and
at least one cable that connects the plurality of electrodes and the reflectance optical sensor to a pulse flowmeter.

2. The structure of claim 1, w reflectance optical sensor is a reflectance pulse oximeter.

3. The structure of claim 1, wherein the optical sensor comprises a light source and optical sensor.

4. The structure of claim 1, wherein the plurality of electrodes are spot electrodes.

5. The structure of claim 1, wherein the plurality of electrodes are semi-circumferential electrodes.

6. The structure of claim 1, wherein the backing is tapered.

7. The structure of claim 1, wherein the plurality of electrodes comprise two electrodes as part of a proximal electrode, and another two electrodes are part of a distal electrode.

8-16. (canceled)

17. A structure for a plethysmogram, the structure comprising:
a backing;
a plurality of electrodes mounted on the backing;
an optical sensor; and
at least one cable that connects the plurality of electrodes and the optical sensor to a pulse flowmeter.

18. The structure of claim 17, wherein the optical sensor is a reflectance pulse oximeter.

19. The structure of claim 17, wherein the optical sensor is a transmission pulse oximeter.

20. The structure of claim 17, wherein the optical sensor comprises a light source and optical sensor.

21. The structure of claim 17, wherein the plurality of electrodes are spot electrodes.

22. The structure of claim 17, wherein the plurality of electrodes are semi-circumferential electrodes.

23. The structure of claim 17, wherein the backing is tapered.

24. The structure of claim 17, wherein the plurality of electrodes comprise two electrodes as part of a proximal electrode, and another two electrodes as part of a distal electrode.
